(19) United States
(12) Patent Application Publication
(10) Pub. No.: US 2005/0101997 A1
(43) Pub. Date: May 12, 2005

(54) Arrangements and methods for determining or treating cardiac abnormalities and inconsistencies

(76) Inventors: Vivek Y. Reddy, Cambridge, MA (US); David J. Milan, Boston, MA (US); Jeremy N. Ruskin, Watertown, MA (US)

Correspondence Address: DORSEY & WHITNEY LLP;INTELLECTUAL PROPERTY DEPARTMENT, 250 PARK AVENUE, NEW YORK, NY, 10177, US

(21) Appl. No.: 10/706,745

(22) Filed: Nov. 12, 2003

(51) Int. Cl. A61N 1/36

(52) U.S. Cl. 607/3

(57) ABSTRACT

An arrangement and method for treating cardiac abnormalities and/or inconsistencies are provided. In particular, a fluid is introduced to a target area within a heart of a subject. Preferably, a volume of the target area which receives the fluid is less than a volume of the heart, and the volume of the target area which receives the fluid is independent from a manner of introducing the fluid to the target area. For example, the volume of the target area which receives the fluid is less than the volume of the heart regardless of whether the fluid is introduced systemically or locally. The arrangement may also include an energy source adapted to transmit energy, such as light, to at least one portion of the target area.

### FIELD OF THE INVENTION

The present invention relates generally to an arrangement and method for treating cardiac abnormalities and inconsistencies in the heart of a subject. In particular, the present invention is directed to an arrangement and method in which a fluid is introduced to a target area within the heart, such that a volume of the target area which receives the fluid is smaller than a volume of the heart, and such that the volume of this target area is independent from a manner of the introduction of the fluid thereto.

### BACKGROUND OF THE INVENTION

Cardiac arrhythmias (such as atrial fibrillation, arrhythmias associated with a scarring of heart tissue, arrhythmias associated with an atrium and/or a ventricle of the heart, etc.) are medical ailments which may affect the performance of the heart. For example, arrhythmias resulting in cardiac arrest are associated with scarring of heart tissue as may occur after the subject experiences a heart attack. In subjects (e.g., human subjects or animals) with normal sinus rhythm, the heart is electrically excited to beat in a synchronous, patterned manner. Nevertheless, in subjects with a cardiac arrhythmia, at least some regions (e.g., abnormal regions) of the heart do not follow the synchronous beating cycle associated with normal conductive heart tissue in for subjects that have a normal sinus rhythm. Specifically, in subjects with a cardiac arrhythmia, the abnormal regions of the heart aberrantly conduct to normal, adjacent regions of the heart, thus disrupting the cardiac cycle of the normal, adjacent region into an asynchronous, cardiac rhythm.

A variety of clinical conditions may arise due to the existence of cardiac arrhythmia. Such clinical conditions may include stroke, heart failure, and thromboembolic events. Conventional arrangements for treating cardiac arrhythmias may include a fluid delivery system, which may be adapted to systemically introduce a photodynamic fluid to the entire heart, and/or to locally introduce the photodynamic fluid to a portion of the heart which includes arrhythmia. For example, the photodynamic fluid can be systemically introduced to the entire heart via a blood vessel, and/or locally introduced to the portion of the heart which includes the arrhythmia via a coronary artery. Generally, the photodynamic liquid increases the sensitivity of cells and/or tissues within the heart to energy. Conventional arrangements can also include an energy source adapted to transmit energy to the portion of the heart which includes the arrhythmia. For example, the energy source may be adapted to transmit energy in the form of light, and the light can have a predetermined wavelength, e.g., between about 350 nm and 700 nm. The predetermined wavelength can be selected such that when the energy is transmitted to those portions of the heart that received the photodynamic fluid, cells and/or tissue associated with those portions of the heart may be damaged or destroyed. Specifically, when the energy is transmitted to those portions of the heart which received the photodynamic liquid, singlet oxygen and/or other reactive species may be generated. In the human body, reactive species such as singlet oxygen are toxic, and can lead to cell and/or tissue destruction.

Nevertheless, in the conventional arrangements, a volume of the heart which receives the photodynamic fluid depends on a manner in which the photodynamic fluid is introduced to the heart, e.g., systemically or locally. In particular, when the photodynamic fluid is systemically introduced, it is delivered to the entire heart. When this is the case, it may be desirable to determine the location of the cardiac arrhythmia before transmitting the energy to the heart. Specifically, the transmission of energy to portions of the heart which received the photodynamic fluid, but which do not include the cardiac arrhythmia, may undesirably damage or destroy cells and/or tissue of the heart that do not include the cardiac arrhythmia. Further, when the photodynamic fluid is locally introduced, it may be desirable to determine the location of the cardiac arrhythmia before introducing the photodynamic fluid. The determination of the location of the cardiac arrhythmia before the introduction of the photodynamic fluid is generally not precise, and is also more difficult than the determination of the location of the cardiac arrhythmia after introducing the photodynamic fluid. Consequently, even when the photodynamic fluid is introduced locally, portions of the heart which do not include the cardiac arrhythmia invariably still disadvantageously receive the photodynamic fluid.

### SUMMARY OF THE INVENTION

Therefore, a need has arisen to provide an arrangement and method for treating cardiac arrhythmia which overcome the above-described and other shortcomings of the related art.

One of the advantages of the present invention is that an arrangement and method are provided to treat cardiac abnormalities by introducing a fluid (e.g., a photodynamic fluid) to a target area (e.g., a scar tissue) within a heart of a subject. Moreover, a volume of the target area which receives the fluid can be less than a volume of the heart, and the volume of the target area which receives the fluid may be independent from the manner (e.g., systemically or locally) of the introduction of the fluid to the target area. Further energy (e.g., energy in the form of light) may be transmitted to the entire heart or to the target area of the heart. Specifically, for the exemplary situation in which only the target area receives the fluid, just the target area may be affected by the energy that is transmitted to the entire heart. Consequently, although the arrangement can be used to determine a location of the target area and to transmit the energy only to such target area, it may be unnecessary to locate the target area prior to transmitting the energy.

According to an exemplary embodiment of the present invention, an arrangement and method to treat a cardiac abnormality (e.g., a cardiac arrhythmia) can introduce the fluid (e.g., a photodynamic fluid, such as a photodynamic compound) to the target area within the heart of the subject. The fluid can be systemically introduced to the target area (e.g., via a blood vessel), locally introduced to the target area (e.g., via a coronary artery), etc. Regardless of whether the fluid is introduced to the target area systemically or locally, the volume of the target area which receives the fluid is preferably smaller than the volume of the entire heart. Also, the volume of the target area which receives the fluid may likely be independent from the manner of the introduction of the fluid to the target area.

The arrangement and method according to another exemplary embodiment of the present invention can also be used to transmit the energy (e.g., light) to the entire heart, the target area and/or a portion of the target area after the fluid is introduced to the target area. For example, when the energy is transmitted to the entire heart, the location of the target area may not necessarily be determined. Preferably, because only the target area receives the fluid, cells and/or tissue associated with the target area would only likely be damaged or destroyed by the energy. Consequently, when the energy is transmitted to the portions of the heart which are outside of the target area, those portions of the heart would likely be unaffected by the energy. When the energy is transmitted only to the target area or to a portion of the target area, the exemplary arrangement and/or method according to the present invention may determine the location of the target area after the fluid is introduced to the target area, but before the energy is transmitted to such target area. For example, the location of the target area may be determined based on at least one predetermined criteria associated with the heart, such as electrical activity within the heart.

In yet another exemplary embodiment of the present invention, the target area may include scar tissue of the heart, e.g., scar tissue generated after the subject experiences a cardiac arrest. Moreover, the scar tissue may have a predetermined metabolic rate, and the liquid may be adapted to be received by only those areas of the heart having a metabolic rate that is greater than or equal to the predetermined metabolic rate, e.g., the target area. In still exemplary embodiment of the present invention, the predetermined metabolic rate associated with the scar tissue of the heart may be greater than the metabolic rate associated with those portions of the heart that are positioned outside of the scar tissue. Further, the liquid may be selected such that the liquid would be concentrated only in the tissues having a metabolic rate which is greater than or equal to the predetermined metabolic rate. As such, likely only the scar tissue, e.g., the target area, may receive the liquid.

### BRIEF DESCRIPTION OF THE DRAWINGS

FIG. 1 is a schematic diagram of an exemplary embodiment of an arrangement according to the present invention for treating a cardiac arrhythmia in a heart of a subject.

FIG. 2 is a perspective view of an exemplary energy source which may be used in the arrangement of FIG. 1.

FIG. 3 is a flow diagram of a first exemplary embodiment of a method according to the present invention for treating the cardiac arrhythmia in the heart of the subject which can be used by the arrangement of FIG. 1.

FIG. 4 is a flow diagram of a second exemplary embodiment of the method according to the present invention for treating the cardiac arrhythmia in the heart of the subject which can also be used by the arrangement of FIG. 1.

### DETAILED DESCRIPTION

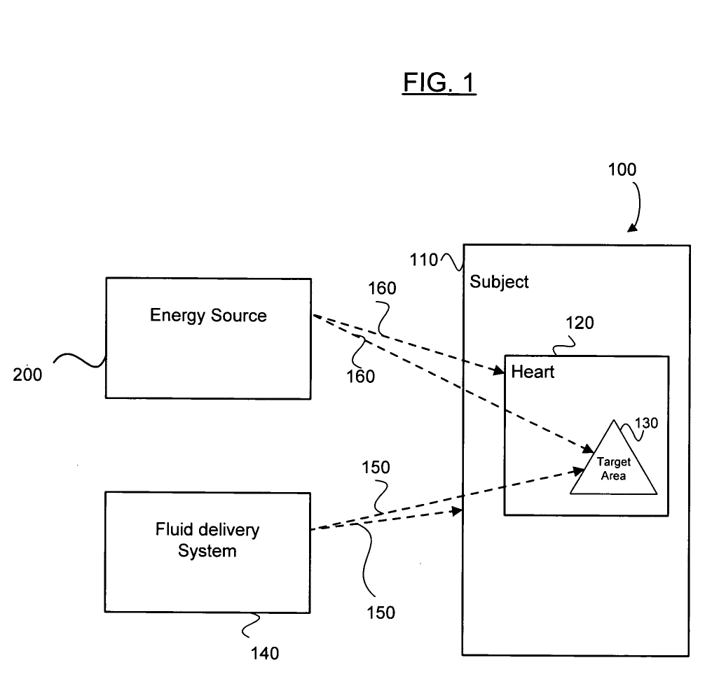

Exemplary embodiments of the present invention and their advantages may be understood by referring to FIGS. 1-4, like numerals being used for like corresponding parts in the various drawings.

Referring to FIG. 1, an exemplary embodiment of an arrangement 100 according to the present invention for treating cardiac abnormalities and inconsistencies is provided. The arrangement 100 may include a fluid delivery system 140. The fluid delivery system 140 may be adapted to introduce a fluid 150 to a target area 130 (e.g., a cardiac arrhythmia) of a heart 120 within a subject 110. For example, the fluid 150 can be delivered systemically, e.g., by injecting the fluid 150 into a vein (not shown) of the subject 110. Alternatively, the fluid 150 can be delivered locally to the target area 130, e.g., via a coronary artery (not shown). In an exemplary embodiment of the present invention, the fluid 150 may be a compound, such as a photodynamic compound or fluid. The photodynamic fluid may include the type of fluids which absorb energy (e.g., energy in the form of light) over a predetermined range of frequencies and produce a chemical reaction, such as, for example, a chemical reaction which produces a toxin or other actor capable of damaging or killing cells and/or tissue. As such, the fluid 150 is adapted to increase the sensitivity of the target area 130 to energy. For example, the predetermined range of frequencies of light can be provided between about 350 nm and 700 nm. Moreover, the cardiac arrhythmia can include atrial fibrillation, arrhythmia associated with scarring of heart tissue, arrhythmia associated with atrium and/or ventricle of the heart, etc. When the fluid 150 is delivered systemically, the location of the target area 130 may (or may not necessarily) be determined before the fluid is delivered systemically. Alternatively, when the fluid 150 is delivered locally to the target area 130, the location of the target area 130 may be determined before delivering the fluid 150 to the target area, e.g., using any imaging technique known to those having ordinary skill in the art.

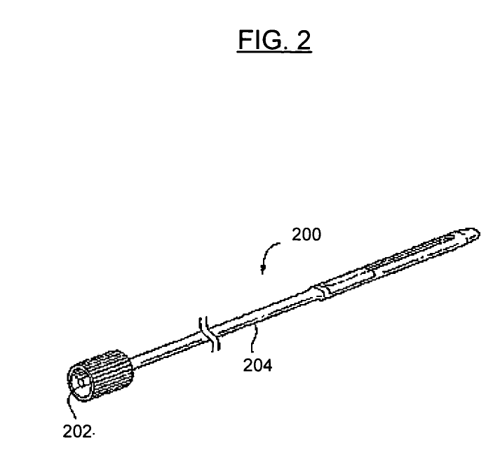

The arrangement 100 of FIG. 1 preferably includes an energy source 200. The energy source 200 may be adapted to transmit a particular amount of energy 160 (e.g., light) to the entire heart 120, the target area 130 of the heart 120 or a portion of the target area 130 after the fluid 150 is introduced into the target area 130. For example, an exemplary embodiment of the energy source 200 according to the present invention for delivering the energy 160 the entire heart 120, the target area 130 or the portion of the target area 130 is shown in FIG. 2. The energy system 200 can include a proximal port 202, which may be adapted to interface with an external light source and/or power supply (not shown). For example, the light source can be a xenon lamp, a high intensity LED source, a laser, and/or any other source adapted to produce an illumination within the predetermined wavelength. The energy source 200 may also include a housing 204, e.g., a flexible housing, which is adapted to allow light to travel from the proximal port 202 to a distal end of the energy source 200 to be output therefrom. The energy source 200 can also include a window or a lens provided at the distal end of the energy source 200. Such window or lens may be adapted to allow the energy 160 to be projected to a desired location, e.g., the entire heart 120, the target area 130 or a portion of the target area 130.

In operation, the fluid delivery system 140 may introduce the fluid 150 to the target area 130. Moreover, regardless of whether the fluid 150 is introduced systemically or locally, the volume of the target area 130 which receives the fluid 150 is preferably smaller than the volume of the entire heart 120. For example, in an exemplary embodiment of the present invention, the target area 130 can include scar tissue, such as the scar tissue generated after the subject 110 experiences a heart attack. The scar tissue may have a predetermined metabolic rate, and the liquid 150 may be selected to have predetermined characteristics for such rate. For example, the predetermined metabolic rate can be greater than the metabolic rate that is associated with normal heart tissue. Moreover, the liquid can be selected such that the liquid concentrates in tissue which has a metabolic rate which is greater than or equal to the predetermined metabolic rate, but does not concentrate in tissue having a metabolic rate which is less than the predetermined metabolic rate. Consequently, when the liquid 150 includes these predetermined characteristics, regardless of whether the liquid 150 is introduced systemically or locally, the liquid 150 may be received by the target area 130, but would likely not be received by those portions of the heart 120 which are outside the target area 130. Due to this fact, when the liquid is introduced systemically, it may be unnecessary to determine the location of the target area 130 prior to systemically introducing the liquid 150. Nevertheless, the system 100 still may be adapted to locally introduce the liquid 150 by determining the approximate location of the target area 130 prior to the introduction of the fluid 150.

After the target site 130 receives the fluid 150, the energy source 200 can transmit the energy 160 to the entire heart 120, the target area 130 or the portion of the target area 130. Specifically, because the target area 130 receives the fluid 150, the transmission of the energy 160 to the target area 130 may generate a chemical reaction which can damage or destroy cells and/or tissue associated with the target area 130. Nevertheless, because those portions of the heart 120 which are outside the target area 130 do not receive the fluid 150, the transmission of the energy 160 to the entire heart 120 may not damage or destroy cells and/or tissue associated with those portions of the heart 120 which are located outside the target area 130. This is because the fluid 150 is not located in such areas, and thus does not react with the energy at those locations.

Consequently, in an exemplary embodiment of the present invention, the arrangement 100 can be used as described above without determining the location of the target area 130. However, to decrease the likelihood that cells and/or tissue associated with the target site 130 are damaged or killed by the energy 160, it may be desirable to determine the location of the target area 130 prior to the transmission of the energy to the subject 110. In this exemplary embodiment, one or more predetermined criteria may be used to determine the location of the target area 130 after the liquid 150 is introduced to the target area 130, and before the energy 160 is transmitted to the subject 110. For example, the electrical activity within the heart 120 can be monitored and analyzed to determine the location of the target area 130. It will be understood by those of ordinary skill in the art that it may be less difficult to determine the location of the target area 130 after the introduction of the fluid 150 to the target area 130 than prior to such introduction of the fluid 150. Moreover, after the target area 130 is located, the energy 160 can be transmitted to the target area 130 or to the portion of the target area 130 instead of to the entire heart 120.

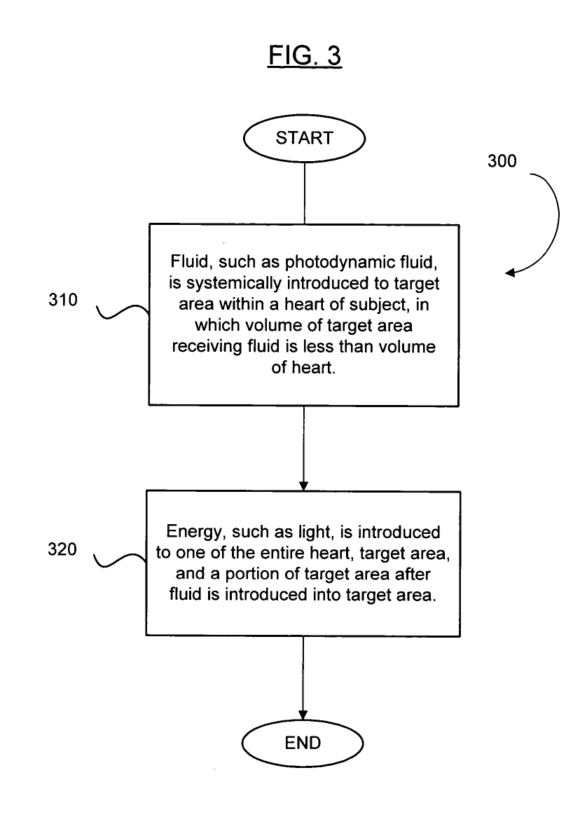

Referring to FIG. 3, a flow diagram of a first exemplary embodiment of a method 300 according to the present invention which can be used by the arrangement 100 of FIG. 1 is depicted. In step 310, the fluid 150, e.g., the photodynamic fluid, is systemically introduced to the target area 130. Moreover, a volume of the area within the heart 120 which receives the fluid 150, i.e., the target area 130, is preferably smaller than a volume of the heart 120. In step 320, the energy 160, such as energy in the form of light, is transmitted to the entire heart 120, the target area 130 or the portion of the target area 130. In this manner, certain abnormalities and/or inconsistencies of the heart 120 can be detected and/or treated.

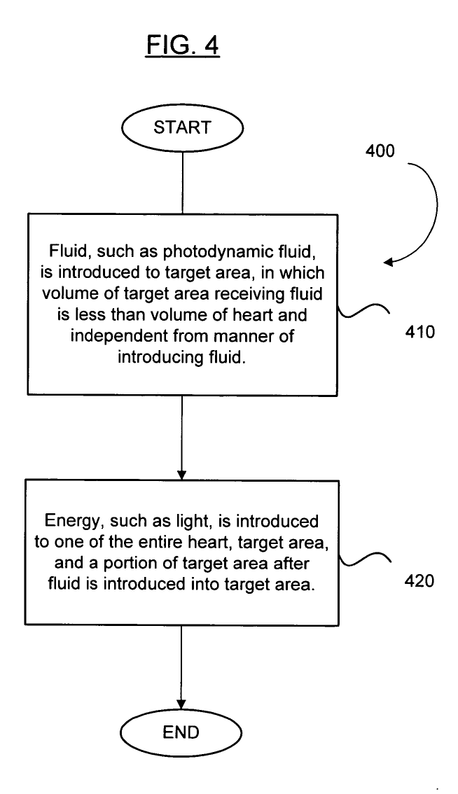

Referring to FIG. 4, a flow diagram of a second exemplary embodiment of a method 400 according to the present invention which can also be used by the arrangement 100 of FIG. 1 is depicted. In step 410, the fluid 150, e.g., the photodynamic fluid, is introduced to the target area 130. Again, the volume of the area within the heart 120 which receives the fluid 150, i.e., the target area 130, is preferably smaller than the volume of the heart 120 independent from a manner in which the fluid 150 is introduced. For example, regardless of whether the fluid 150 is introduced systemically or locally, the volume of the area within the heart 120 which receives the fluid 150, i.e., the target area 130, is smaller than the volume of the entire heart 120. In step 420, similar to step 320 of FIG. 3, the energy 160, such as energy in the form of light, is transmitted to the entire heart 120, the target area 130 or the portion of the target area 130.

While the invention has been described in connecting with preferred embodiments, it will be understood by those of ordinary skill in the art that other variations and modifications of the preferred embodiments described above may be made without departing from the scope of the invention. Other embodiments will be apparent to those of ordinary skill in the art from a consideration of the specification or practice of the invention disclosed herein. It is intended that the specification and the described examples are considered as exemplary only, with the true scope and spirit of the invention indicated by the following claims.

1. A method for detecting or treating at least one of cardiac abnormalities and cardiac inconsistencies, comprising the steps of:
systemically introducing a fluid to a target area of a heart of a subject, wherein a volume of the target area which receives the fluid is less than a volume of the heart; and
transmitting energy to at least one portion of the target area.

2. The method of claim 1, wherein the fluid is a compound.

3. The method of claim 2, wherein the compound is a photodynamic compound.

4. The method of claim 1, wherein the step of transmitting energy comprises the substep of transmitting the energy to the entire target area.

5. The method of claim 1, wherein the step of transmitting energy comprises the substep of transmitting the energy to the entire heart.

6. The method of claim 1, wherein at least one of the cardiac abnormalities is a cardiac arrhythmia.

7. The method of claim 1, wherein the energy transmitted to the at least one portion of the target area comprises light.

8. The method of claim 1, wherein the target area comprises scar tissue.

9. The method of claim 8, wherein the scar tissue has a predetermined metabolism, and wherein the liquid is adapted to be received only by those areas of the heart having a metabolism which is greater than or equal to the predetermined metabolism.

10. The method of claim 1, wherein the liquid increases a sensitivity of the target area for energy such that the transmission of energy to the at least one portion of the target area damages at least one of a plurality of cells and a tissue within the target area.

11. A method for detecting or treating at least one of cardiac abnormalities and cardiac inconsistencies, comprising the steps of:
introducing a fluid to a target area within a heart of a subject, wherein a volume of the target area which receives the fluid is less than a volume of the heart, and wherein the volume of the target area which receives the fluid is independent from a manner of the introduction of the fluid to the target area; and
transmitting energy to at least one portion of the target area.

12. The method of claim 11, wherein the introducing step comprises the substep of systemically introducing the fluid to the target area.

13. The method of claim 11, wherein the introducing step comprises the substep of locally introducing the fluid to the target area.

14. The method of claim 13, wherein the step of locally introducing comprises the substep of introducing the fluid to the target area via a coronary vessel.

15. The method of claim 11, wherein the fluid is a compound.

16. The method of claim 15, wherein the compound is a photodynamic compound.

17. The method of claim 11, wherein the step of transmitting energy comprises the substep of transmitting the energy to the entire target area.

18. The method of claim 16, wherein the step of transmitting energy further comprises the substep of determining a location of the target area based on at least one predetermined criteria associated with the heart prior to the transmission of the energy to the entire target area.

19. The method of claim 17, wherein the at least one predetermined criteria comprises electrical activity within the heart.

20. The method of claim 11, wherein the step of transmitting energy comprises the substep of transmitting the energy to the entire heart.

21. The method of claim 19, wherein the energy is transmitted to the entire heart without determining a location of the target area.

22. The method of claim 11, wherein the cardiac abnormality is a cardiac arrhythmia.

23. The method of claim 11, wherein the energy transmitted to the at least one portion of the target area comprises light.

24. The method of claim 11, wherein the target area comprises scar tissue.

25. The method of claim 23, wherein the scar tissue has a predetermined metabolism, and wherein the liquid is adapted to be received by only those areas of the heart having a metabolism which is greater than or equal to the predetermined metabolism.

26. The method of claim 11, wherein the liquid increases a sensitivity of the target area to energy such that the transmission of energy to the at least one portion of the target area damages at least one of a plurality of cells and a tissue within the target area.

27. An arrangement for detecting or treating at least one of cardiac abnormalities and cardiac inconsistencies, comprising:
a fluid delivery system adapted to systemically introduce a fluid to a target area of a heart of a subject, wherein a volume of the target area which receives the fluid is less than a volume of the heart; and
an energy source adapted to transmit energy to at least one portion of the target area.

28. The arrangement of claim 26, wherein the fluid is a compound.

29. The arrangement of claim 27, wherein the compound is a photodynamic compound.

30. The arrangement of claim 26, wherein the energy source is further adapted to transmit the energy to the entire target area.

31. The arrangement of claim 26, wherein the energy source is further adapted to transmit the energy to the entire heart.

32. The arrangement of claim 26, wherein the cardiac abnormality is a cardiac arrhythmia.

33. The arrangement of claim 26, wherein the energy transmitted to the at least one portion of the target area comprises light.

34. The arrangement of claim 26, wherein the target area comprises scar tissue.

35. The arrangement of claim 33, wherein the scar tissue has a predetermined metabolism, and wherein the liquid is adapted to be received only by those areas of the heart having a metabolism which is greater than or equal to the predetermined metabolism.

36. The arrangement of claim 26, wherein the liquid increases a sensitivity of the target area to energy such that the transmission of energy to the at least one portion of the target area damages at least one of a plurality of cells and a tissue within the target area.

37. An arrangement for detecting or treating at least one of cardiac abnormalities and cardiac inconsistencies, comprising:
a fluid delivery system adapted to introduce a fluid to a target area within a heart of a subject, wherein a volume of the target area which receives the fluid is less than a volume of the heart, and wherein the volume of the target area which receives the fluid is independent from a manner of introducing the fluid to the target area; and
an energy source adapted to transmit energy to at least one portion of the target area.

38. The arrangement of claim 36, wherein the fluid delivery system is adapted to systemically introduce the fluid to the target area.

39. The arrangement of claim 36, wherein the fluid delivery system is adapted to locally introduce the fluid to the target area.

40. The arrangement of claim 38, wherein the fluid delivery system is further adapted to locally introduce the fluid to the target area via a coronary vessel.

41. The arrangement of claim 36, wherein the fluid is a compound.

42. The arrangement of claim 40, wherein the compound is a photodynamic compound.

43. The arrangement of claim 36, wherein the energy source is further adapted to transmit the energy to the entire target area.

44. The arrangement of claim 42, wherein the energy source is further adapted to determine a location of the target area based on at least one predetermined criteria associated with the heart prior to transmitting the energy to the entire target area.

45. The arrangement of claim 43, wherein the at least one predetermined criteria comprises electrical activity within the heart.

46. The arrangement of claim 36, wherein the energy source is further adapted to transmit the energy to the entire heart.

47. The arrangement of claim 45, wherein the energy is transmitted to the entire heart without determining a location of the target area.

48. The arrangement of claim 36, wherein the cardiac abnormality is a cardiac arrhythmia.

49. The arrangement of claim 36, wherein the energy transmitted to the at least one portion of the target area comprises light.

50. The arrangement of claim 36, wherein the target area comprises scar tissue.

51. The arrangement of claim 49, wherein the scar tissue has a predetermined metabolism, and wherein the liquid is adapted to be received by only those areas of the heart having a metabolism which is greater than or equal to the predetermined metabolism.

52. The arrangement of claim 36, wherein the liquid increases a sensitivity of the target area to energy such the transmission of energy to the at least one portion of the target area damages at least one of a plurality of cells and a tissue within the target area.
